(19) United States
(12) Patent Application Publication
(10) Pub. No.: US 2024/0238442 A1
(43) Pub. Date: Jul. 18, 2024

(54) LIPOSOMES DISPLAYING A GLYCAN LIGAND FOR CD33 AND THEIR USE IN THE TREATMENT OF ALZHEIMER'S DISEASE

(71) Applicant: THE GOVERNORS OF THE UNIVERSITY OF ALBERTA, Edmonton, Alberta (CA)

(72) Inventors: Matthew MACAULEY, Edmonton, Alberta (CA); Abhishek BATTACHERJEE, Edmonton, Alberta (CA); Arjun Singh BAINS, Edmonton, Alberta (CA); Gour DASKHAN, Edmonton, Alberta (CA)

(21) Appl. No.: 18/558,999

(22) PCT Filed: May 5, 2021
(86) PCT No.: PCT/CA2021/050629

§ 371 Date: Nov. 3, 2023

(51) Int. Cl. A61K 47/69; A61K 9/00; A61K 47/61

(52) U.S. Cl. CPC A61K 47/6911; A61K 9/0085; A61K 47/61

(57) ABSTRACT

The present application provides a liposomal composition that multivalently displays a specific glycan ligand, or combination of specific glycan ligands, to specifically target CD33 on microglial cells. Multivalent engagement of CD33 with glycan ligands in the liposomal composition are useful in modulating microglial cell function, particularly as a therapeutic for the preventing, treating or delaying progression of Alzheimer's disease.

### FIELD OF THE INVENTION

The present application pertains to the field of liposomal compositions for therapeutic use. More particularly, the present application relates to liposomal compositions displaying a ligand for CD33, and methods of manufacture and uses thereof in the treatment of Alzheimer's disease.

### INTRODUCTION

Alzheimer's disease (AD) is a slowly progressive and ultimately fatal degenerative brain disorder. It is the most prevalent chronic neurodegenerative disease in the world, and is becoming even more prevalent as a result of increased longevity and global population aging. Currently, there is no cure for AD and treatments merely function to temporarily ameliorate symptoms of memory loss and improve behavioral disturbances; no disease modifying treatment is yet available.

The brains of AD patients are characterized by extracellular accumulation of amyloid plaques, composed of 3-amyloid peptide (Aβ), and intraneuronal accumulation of intracellular neurofibrillary tangles, composed primarily of aggregated forms of hyperphosphorylated tau protein. Consequently, a great deal of research has focused on modulators of amyloid production pathways, including monoclonal antibodies against Aβ, and on disrupting tau pathology. To date, none have been particularly successful.

Recently, genetic approaches and computational strategies have converged on immunoregulation events as key to the pathogenesis of AD and a number of genes have been highly linked to the onset and development of AD. Many of the genes associated with AD susceptibility are involved in microglial biology.¹ As the major immune cell in the brain, microglia play a myriad of roles and are connected with AD progression through their ability to phagocytose Aβ.²

Mutation and/or differential expression of the gene encoding glycan-binding protein, CD33, has been linked to AD susceptibility.³ The link between CD33 and AD susceptibility was identified through genome-wide association studies, where a rare allele of CD33 is AD protective.^{4,5} This rare allele leads to alternative mRNA splicing,⁶ leading to reduced expression of a long isoform of CD33, called CD33M, and increased expression of a short isoform, called CD33m. Disentangling the individual roles of these two CD33 isoforms has been challenging since human cells express a mixture of these two CD33 isoforms. Moreover, CD33 in mice has several highly divergent features and does not undergo alternative mRNA splicing.⁷ Nevertheless, significant progress has recently been made and supports a role for CD33M in repressing phagocytosis, while distinct roles for CD33m have been also identified recently.⁷⁻¹⁰

Therapeutic targeting of proteins implicated in AD susceptibility has become an attractive approach. For example, antibodies targeting TREM2 have undergone rigorous evaluation in mouse models.¹¹⁻¹³ These results have pointed to stimulation of microglial cell proliferation as a key means of skewing microglia into a protective phenotype.¹³ Anti-TREM2 antibodies are now under clinical evaluation (NCT03635047). TREM2 is proposed to be negatively-regulated by CD33,^{14,15} and antibodies targeting CD33—which have a long history in the clinic for the treatment of leukemia¹⁶—are being assessed as means of modulating microglia. It has been demonstrated that anti-CD33 antibodies deplete CD33M from the surface, which is presumably where it needs to be in order to repress phagocytosis.^{17,18} However, as murine CD33 does not serve as a good model for human CD33, in vivo preclinical testing of anti-CD33 antibodies has not been carried out. Despite this gap, anti-CD33 antibodies have entered clinical trials (NCT03822208).

CD33 is a member of sialic acid-binding immunoglobulin-type lectin (Siglec) family of immunomodulatory receptors.¹⁹ CD33 binds to both α2-3 and α2-6 linked sialosides, with emerging evidence that binding may be enhanced by underlying sulfation.^{20,21} Engaging Siglecs with high-affinity and specific glycan ligands is an alternative approach to antibody targeting.²² A high-affinity and select glycan ligand for targeting human CD33 was previously developed through dual modification of the 5- and 9-position of sialic acid with hydrophobic substitutents.²³ Recently, a high-resolution crystal structure of CD33 and this high-affinity CD33 ligand was determined, providing a basis for how the modifications exploit surrounding hydrophobic pockets.²⁴ Moreover, microparticles displaying this CD33 ligand was observed to increase uptake of Aβ peptide in human monocytic cells (THP1) in vitro, although the mechanism by which this occurred was not elucidated.

An urgent need remains for effective therapies for AD that target disease progression or prevention, rather than merely providing an alleviation of symptoms.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide liposomal compositions and methods for treatment of Alzheimer's Disease. In accordance with an aspect of the present application, there is provided a liposomal composition for inducing microglial phagocytosis, said composition comprising a liposome nanoparticle and a glycan ligand for CD33 displayed on the liposome nanoparticle.

In some embodiments, the glycan ligand is present at an amount of greater than 1 mol %, preferably from 1 mol % to 5 mol %, more preferably from 3 mol % to 5 mol %, or most preferably about 3.3 mol %. CD33 binds the glycan ligand more strongly than it binds its natural sialic acid ligands. For example, CD33 has a binding affinity for the glycan ligand that is at least about 20 times, preferably at least about 25 times, or more preferably at least about 30 times, stronger than the binding affinity of CD33 for its natural sialic acid ligands. In some embodiments, the glycan ligand has a dissociation constant (K_{D}), based on monovalent binding of the glycan ligand with CD33, that is less than 120 μM, less than 110 μM, less than 100 μM, or preferably less than 90 μM.

In one embodiment, the liposomal composition comprises a glycan ligand formed by lipid attachment at the amine of the compound of formula 1

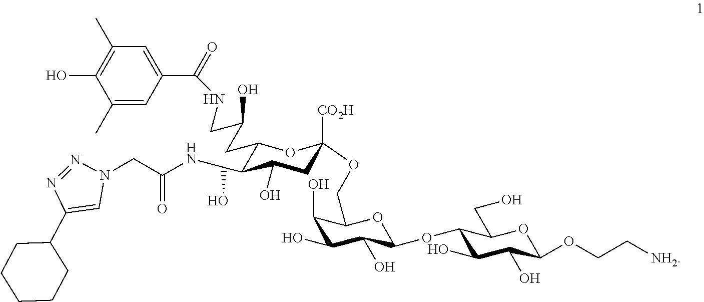

In accordance with another aspect of the present application, there is provided a method for preventing, treating or delaying the progression of Alzheimer's disease in a subject, said method comprising administering to the subject a liposomal composition comprising a liposome nanoparticle and a glycan ligand for CD33 displayed on the liposome nanoparticle, as described herein. In some embodiments, the liposomal composition is administered in an amount effective to induce microglial phagocytosis in the subject, whereby the level of microglial phagocytosis in the subject is increased in comparison to that occurring in the untreated subject.

In accordance with some embodiments, the liposomal formulation is administered directly to the brain of the subject, for example, by intracerebroventricular injection or intraparenchymal injection.

In accordance with another aspect of the present application, there is provided a use of a liposomal composition for inducing microglial phagocytosis in a subject, said composition comprising a liposome nanoparticle and a glycan ligand for CD33 displayed on the liposome nanoparticle, as described herein. In accordance with some embodiments, the use is for prevention, treatment or delay of progression of Alzheimer's disease in the subject.

### BRIEF DESCRIPTION OF FIGURES

For a better understanding of the application as described herein, as well as other aspects and further features thereof, reference is made to the following description which is to be used in conjunction with the accompanying drawings, where:

FIG. 1: Fluorophore conjugated CD33L liposome formulation for engaging CD33 on cells. (a-c) Chemical structures of CD33 ligand (CD33L; (a)), Alexa Fluor 647 (AF647; (b)), and pH-sensitive rhodamine (pHrodo; (c)) conjugated to DPSE through 2000 MW (45 repeats, on average) PEG. (d,e) Cartoon diagrams of CD33L (d) and naked (e) liposomes. (f) Binding of naked (black) and CD33L liposomes (grey) to CD33^{-/-} (middle panel), and CD33^{-/-} U937 cells transduced with WT CD33M (middle panel) or R119A CD33M (right panel). (g) Binding of naked (black) and CD33L liposomes (grey) to primary microglia isolated from WT (Cx₃cre^{Cre-/+} hCD33M^{-/-}) or hCD33M transgenic (Cx₃cre^{Cre-/+} hCD33M^{-/+}) mice. Binding of liposomes to cells was measured by total AF647 signal by flow cytometry.

FIG. 2: CD33L liposomes increase phagocytosis of polystyrene beads in U937 cells. (a,b) Liposomal nanoparticles were formulated at different CD33L densities and tested for their ability to modulate phagocytosis. (c,d) The most efficient formulation (3.33% CD33L) was selected and tested at different liposomal concentrations. (e,f) A liposomal concertation of 100 μM was selected and incubated with cells for different amount of times prior to examining phagocytosis. Phagocytosis was measured by flow cytometry (a,c,e) and the percentage of phagocytic cells were quantified (b,d,f). Statistical significance was calculated using an unpaired Student's T-test.

FIG. 3: CD33L liposomes induce internalization of CD33. (a-f) CD33L liposomes formulated with different CD33L densities, containing pHrodo™ and Alexa 647, were incubated with WT U937 cells. (g-l) CD33L liposomes formulated with 3.3 mol % CD33L were incubated with WT U937 cells at different concentrations. (m-r) CD33L liposomes were formulated with 3.3 mol % CD33L and incubated with U937 cells at 100 μM for different amounts of time. Cell surface CD33M cell surface staining (a,b,g,h,m,n), internalized liposomes (c,d,i,j,o,p), and total liposome binding (e,f,k,l,q,r) measured by flow cytometry. Raw flow cytometry plots (a,c,e,g,i,k,m,o,q) and summary plots of the MFI values (b,d,f,h,j,l,n,p,r) are gradient coded for the conditions used. Assays were replicated three times and statistical significance calculated based on an unpaired Student's T-test.

FIG. 4: Increased phagocytosis by CD33L liposomes is dependent on the glycan-binding and signaling abilities of CD33. Naked (black) or CD33L (grey) liposomes were incubated with CD33^{-/-} U937 cells transduced with (a,b) empty vector, (c,d) WT CD33M, (e,f) R119A CD33M, (g,h) Y340A CD33M, (i,j) and Y358A CD33M prior to performing phagocytosis assay. Raw flow cytometry plots (a,c,e,g,i) and summary plots of the MFI values (b,d,f,h,j) are shown for each cell type. (k) CD33M internalization following incubation with naked liposomes or CD33L liposomes was examined on CD33^{-/-} U937 cells transduced with WT CD33M, Y340A CD33M or Y358A CD33M. Assay were replicated three times and Statistical significance calculated based on an unpaired Student's T-test.

FIG. 5. CD33L liposomes enhance phagocytosis in hCD33M transgenic mouse microglia. (a) Schematic for a competitive phagocytosis assay with primary microglia taken from WT (black) and hCD33M transgenic (grey) mice. (b) Gating strategy for the competitive phagocytosis assay. (c-h) Results of the competitive flow cytometry-based uptake of polystyrene microbeads. Isolated microglia were pre-treated with PBS (c,d), Naked Liposome (e,f), or CD33L Liposomes (g,h). Raw flow cytometric data (c,e,g) and the percentage of phagocytosis was quantified in each condition (d,f,h). Each point represents an independent experiment from different mice. N.S. represents no statistical significance based on a paired Student's T-test.

FIG. 6: Intracerebroventricular injection (ICV) of CD33L liposomes modulates phagocytosis of hCD33M microglia. (a) Schematic representation of the study, in which WT (black) or hCD33M transgenic mice (grey) were administered PBS, naked liposomes, or CD33L liposomes, followed by mixing of microglia, and a competitive phagocytosis assay. (b,c) AF647 of microglia after injection of naked or CD33L liposomes. (d-i) Results of the competitive flow cytometry-based uptake of aggregated Aβ₁₋₄₂. WT (black) and hCD33M transgenic mice (grey) in mice injected with PBS (d,e), naked Liposome (f,g), or CD33L liposomes (h,i). Raw flow cytometric data (d,f,h) and the percentage of phagocytosis was quantified in each condition (e,g,i). Each point represents an independent experiment from different mice. N.S. represents no statistical significance based on a paired Student's T-test.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “liposomal composition” refers to a complex that contains a lipid component that forms a bilayer liposome structure. It is typically a semi-solid, ultra-fine vesicle sized between about 10 and about 200 nanometers, or more particularly between about 80 to about 160 nm. The liposomal composition displays on or incorporates into the lipid component a binding moiety, in particular a glycan ligand, that is specific for a target molecule (e.g., a Siglec) on a target cell. The term “displayed on” is used herein to reference the ligand that forms part of the liposomal composition and is available for binding its target, irrespective of whether it is displayed on or incorporated or integrated into the lipid component.

As used herein, the term “contacting” has its normal meaning and refers to combining two or more agents (e.g., compositions or small molecule compounds) or combining agents with cells. Contacting can occur in vitro, e.g., combining an agent with a cell or combining two cells in a test tube or other container. Contacting can also occur in vivo, e.g., by targeted delivery of an agent to a cell inside the body of a subject.

As used herein, the term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

As used herein, the term “treating” includes the administration of compounds or compositions to a subject to prevent or delay the onset of the symptoms, complications, or biochemical indicia of a disease, especially AD, alleviating the symptoms or arresting or inhibiting further development of the disease, condition, or disorder. Subjects in need of treatment include those already suffering from the disease or disorder as well as those being at risk of developing the disorder.

Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, the term “effective amount” of a substance is an amount sufficient to produce a desired effect. In some embodiments, an effective amount of a vector is an amount sufficient to infect a sufficient number of target cells of a target tissue of a subject. The effective amount will depend on a variety of factors such as, for example, the species, age, weight, health of the subject, and the mode or site of administration, and may thus vary among subjects and administrations.

CD33 is a sialic acid binding Ig-like lectin (Siglec) that functions as an immunomodulatory receptor and is expressed throughout the myeloid lineage of the immune system, including microglia in brain. The established role for the major CD33 isoform in suppressing microglial cell phagocytosis and the strong genetic link between CD33 and Alzheimer's disease (AD) susceptibility makes it an attractive therapeutic target.

The present inventors have surprisingly found that liposomes engineered to specifically bind CD33 are useful in modulating microglial cell function to increase phagocytosis. In particular, the present application provides liposomes that multivalently display a specific glycan ligand, or combination of specific glycan ligands, of CD33 (referenced herein as a “CD33L liposome” or “CD33L liposomal composition”) to target CD33. Multivalent engagement of CD33 with glycan ligands in the CD33L liposomes can modulate microglial cell function and are useful as a therapeutic for the preventing, treating or delaying progression of AD. In particular, the CD33L liposomal composition increases microglial phagocytosis, in comparison to the microglial phagocytosis in untreated cells, which is useful for clearance of amyloid-β peptide, for example, from the brain (e.g., brain parenchyma) of patients with senile plaques—a pathological feature of Alzheimer's Disease.

### Glycan Ligands

Glycan ligands of CD33 are compounds that are specifically recognized and bound by CD33. “Specific” recognition and binding means that the binding pair exhibit binding with each other under conditions where they do not bind to another molecule or only minimally bind to another molecule(s), for example, by non-specific interactions. In accordance with the present invention, the glycan ligand present on the liposomal composition specifically recognizes and binds to CD33.

The glycan ligands can be natural or synthetic ligands that have a high affinity for CD33. As used herein, “high affinity” is intended to indicate that the binding of the ligand by CD33 occurs with a higher affinity than the binding by CD33 of its natural sialic acid ligands. The term “natural sialic acid ligands” is intended to reference the ligands that are normally recognized and bound by CD33 in mammals.

In accordance with certain embodiments, the binding affinity of CD33 to the high affinity glycan ligand is at least 20 times stronger, or at least 25 times stronger or, more preferably at least about 30 times stronger than the binding affinity of CD33 to its natural ligand.

In accordance with some embodiments, the ligand, or ligands, is/are selected to have a dissociation constant (K_{D}), based on one to one (monovalent) binding with CD33, that is less than 120 μM, less than 110 μM, less than 100 μM, or preferably less than 90 μM. However, each CD33L liposome is multivalent such that it displays a plurality of ligands. Consequently, the CD33L liposome binding to cells expressing CD33 takes advantage of the multiple binding interactions of the CD33L liposome to the CD33 receptors on the surface of these cells, which results in selective binding with high avidity, for example with sufficiently high avidity to induce internalization of CD33 and/or to enhance phagocytosis by the cells expressing CD33. The “velcro” effect from the multivalency of the CD33L liposomes allows the plurality of relatively weak interactions to provide a strong additive binding.

Typically, the glycan ligand used in the liposomes of the present application comprise homo- or heteropolymers of monosaccharide residues. In particular, the glycan ligand can be a derivative of, or contain, an α-2-6-linked sialic acid. The CD33L liposomes can display a single glycan ligand type or can comprise a mixture of different glycan ligands.

Examples of various Siglec glycan ligands that can be used in the CD33L liposomes of the present application are reported in the literature, e.g., Paulson et al., WO 2007/056525; Blixt et al., J. Am. Chem. Soc. 130:6680-1, 2008, Movsisyan and Macauley, Org. Biomol. Chem. 18(30): 5784-5797, 2020, each of which are incorporated by reference in their entirety.

The glycan ligand can be used directly in surface modification of a preformed liposome or it can be coupled to a lipid moiety and then used in the formation of the CD33L liposome. For incorporation of the glycan ligand in the liposome during liposome formation or via a micelle, the glycan ligands can be modified to include a lipid moiety to facilitate the integration of the ligand in the liposome bilayer. Such a modification can be achieved by directly binding a lipid moiety to the glycan ligand. Alternatively, the lipid moiety is connected to the glycan ligand via a linker. In some examples, the linker molecule comprises polyethylene glycol (PEG). However, a PEG linker is not required and may, in some instances, be contraindicated when the CD33L liposome is intended to be formulated to cross the blood-brain barrier.

In accordance with one embodiment, the glycan ligand has the structure of compound 1, which may be directly bound to the surface of a liposome to form the CD33L liposome. Alternatively, the glycan ligand having the structure of compound 1 can be connected to a PEGylated lipid moiety to form compound 3, with the PEG linker, for incorporation in the liposome, as illustrated below.

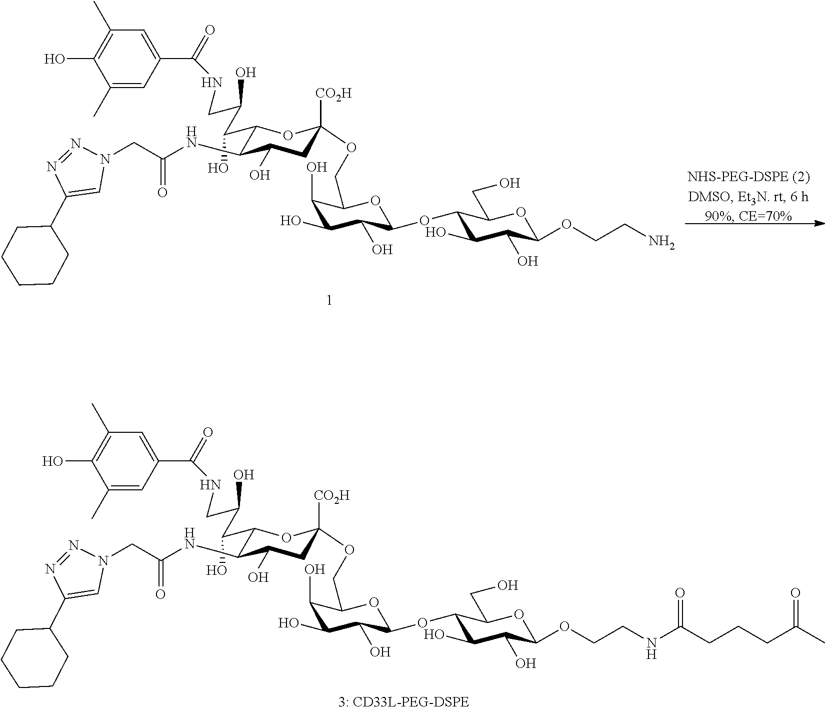

Use of liposomes displaying the above ligand and an antigen were useful in inhibiting antigen-IgE mediated mast cell/basophil activation and degranulation. Interestingly, however, the liposomes that included only the above ligand, without the antigen, did not modulate mast cell/basophil activation and degranulation even though they did bind to the cells (WO 2018/009825 and Duan, S et al., J Clin Invest, 129 (3):1387, 2019).

### Liposomal Composition

The liposome component of the composition of the present application is typically a vesicular structure of a water-soluble particle obtained by aggregating amphipathic molecules including a hydrophilic region and a hydrophobic region. While the liposome component is a closed micelle formed by any amphipathic molecules, it preferably includes lipids. For example, the liposomes of the invention exemplified herein contain phospholipids such as distearoyl phosphatidylcholine (DSPC) and polyethyleneglycol-distearoyl phosphoethanolamine (PEG-DSPE). Other phospholipids can also be used in preparing the liposomes of the invention, including dipalmitoylphosphatidylcholine (DPPC), dioleylphosphatidylcholine (DOPC) and dioleylphosphatidyl ethanolamine (DOPE), sphingoglycolipid and glyceroglycolipid. These phospholipids are used for making the liposome, alone or in combination of two or more or in combination with a lipid derivative where a non-polar substance such as cholesterol or a water-soluble polymer such as polyethylene glycol has been bound to the lipid.

The liposomal compositions can be prepared in accordance with methods well known in the art. For example, incorporation of a CD33 ligand on the surface of a liposome can be achieved by any of the routinely practiced procedures. Detailed procedures for producing a liposome nanoparticle bearing a binding moiety are also exemplified in the Examples provided herein. These include liposomes bearing an incorporated glycan ligand (e.g., compound 1).

In addition to the methods and procedures exemplified herein, various methods routinely used by the skilled artisans for preparing liposomes can also be employed in the present invention. For example, the methods described in Chen et al., Blood 115:4778-86, 2010; Liposome Technology, vol. 1, 2nd edition (by Gregory Gregoriadis (CRC Press, Boca Raton, Ann Arbor, London, Tokyo), Chapter 4, pp 67-80, Chapter 10, pp 167-184 and Chapter 17, pp 261-276 (1993), Bednar et al., J Vis. Exp. 140:58285, 2018, and WO 2018/009825 can be used. More specifically, suitable methods include, but are not limited to, a sonication method, an ethanol injection method, a French press method, an ether injection method, a cholic acid method, a calcium fusion method, a lyophilization method and a reverse phase evaporation method.

The size of the CD33L liposome of the present invention is not particularly limited, and typically the average particle size is from 10 to 200 nm, preferably from 50 to 180 nm or more preferably from 80 to 160 nm, on average. Typically, liposomes are measured to assess their particle size using dynamic light scattering after extrusion. In one example, CD33L liposomes assessed in this manner were found, on average, to have a particle size of 120+/−20 nm.

The structure of the liposome is not particularly limited, and may be any liposome such as unilamella and multilamella. As a solution encapsulated inside the liposome, it is possible to use buffer and saline and others in addition to water.

The CD33L liposome of the present invention comprises sufficient glycan ligand to provide effective binding to CD33. The amount of glycan ligand incorporated in the CD33L liposome will be dependent, at least in part, on the binding affinity of the ligand to CD33: the stronger the binding affinity, the less glycan ligand is required. In accordance with some embodiments, the CD33L liposome comprises the glycan ligand at an amount of at least 0.1 mol % (based on the total composition of the lipids liposome). Preferably, the CD33L liposome comprises the glycan ligand at an amount of at least 0.5 mol %, or at an amount of at least 1%, from about 1 mol % to about 5 mol %, or from about 3 mol % to about 5 mol %. In some embodiments, the CD33L liposome comprises about 3.3 mol % of the glycan ligand.

In the embodiments in which PEGylated lipids are employed in the production of the CD33L, PEG is present at an amount of from about 2 mol % to about 15 mol %, or from about 2 mol % to about 10 mol %, or, preferably, about 5 mol %.

### Pharmaceutical Composition and Method of Use

The present inventors have surprisingly found that the liposomal composition comprising the specific glycan ligand for CD33 is effective in increasing phagocytosis of microglia in the brain. As a result, the liposomal composition of the present invention is useful in the treatment, prevention or delay of progression of AD, for example, by targeting Aβ plaque accumulation.

Accordingly, the provided herein is a method for preventing, treating or delaying progression of AD in a subject, said method comprising administering the CD33L liposomal composition to the subject.

Where clinical application of the CD33L liposomal composition is undertaken, it is necessary to prepare the liposome complex as a pharmaceutical composition appropriate for the intended application.

Pharmaceutical compositions of the invention comprise an effective amount of the liposomal compositions formulated with at least one pharmaceutically acceptable carrier. Pharmaceutical compositions of the invention can be prepared by methods well known in the art of pharmacy. See, e.g., -Goodman & Gilman's The Pharmacological Bases of Therapeutics, Hardman et al., eds., McGraw-Hill Professional (10th ed., 2001); Remington: The Science and Practice of Pharmacy, Gennaro, ed., Lippincott Williams & Wilkins (20th ed., 2003); and Pharmaceutical Dosage Forms and Drug Delivery Systems, Ansel et al. (eds.), Lippincott Williams & Wilkins (7th ed., 1999). In addition, the pharmaceutical compositions of the invention may also be formulated to include other medically useful drugs or biological agents or to be administered together with other medically useful drugs or biological agents via the same or different routes of administration. When administered “together,” the pharmaceutical composition of the present invention is administered at simultaneously with, before or after administration of the other medically useful drugs or biological agents.

For in vivo applications, the liposomal compositions set forth herein can be administered to a subject in need of treatment according to protocols already well established in the art. The liposomal compositions can be administered alone or in combination with a carrier in an appropriate pharmaceutical composition. Typically, a therapeutically effective amount of the liposomal compositions is combined with a pharmaceutically acceptable carrier. The pharmaceutically acceptable carrier is any carrier known or established in the art. Exemplary pharmaceutically acceptable carriers include sterile pyrogen-free water and sterile pyrogen-free saline solution.

Other forms of pharmaceutically acceptable carriers that can be utilized for the present invention include binders, disintegrants, surfactants, absorption accelerators, moisture retention agents, absorbers, lubricants, fillers, extenders, moisture imparting agents, preservatives, stabilizers, emulsifiers, solubilizing agents, salts which control osmotic pressure, diluting agents such as buffers and excipients usually used, depending on the use form of the formulation. These are optionally selected and used depending on the unit dosage of the resulting formulation and depending on the route of administration.

The phrases “pharmaceutically” or “pharmacologically acceptable” refer to compositions that do not produce an adverse, allergic or other untoward reaction when administered to the subject (an animal or a human). As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the liposomal composition of the present invention, its use in the therapeutic compositions is contemplated. As noted above, supplementary active ingredients also can be incorporated into the compositions.

The therapeutic compositions of the present embodiments may include classic pharmaceutical preparations. Administration of therapeutic compositions according to the present invention will be via any common route so long as the target tissue is available via that route. In the present case, administration to the brain is required. This can be achieved via direct administration, for example using intracerebroventricular (ICV) injection, intracranial or intrathecal injection. Alternatively, the liposomal composition can be specifically formulated to allow the composition to cross the blood-brain barrier. In either alternative, such compositions would normally be administered as pharmaceutically acceptable compositions that include physiologically acceptable carriers, buffers or other excipients.

An effective amount of the therapeutic liposomal composition is determined based on the intended goal. The term “unit dose” or “dosage” refers to physically discrete units suitable for use in a subject, each unit containing a predetermined-quantity of the therapeutic liposomal composition calculated to produce the desired responses, in association with its administration, i.e., the appropriate route and treatment regimen. The quantity to be administered, both according to number of treatments and unit dose, depends on the protection desired.

To gain a better understanding of the invention described herein, the following examples are set forth. It should be understood that these examples are for illustrative purposes only. Therefore, they should not limit the scope of this invention in any way.

### EXAMPLES

### Example 1: Increasing Phagocytosis of Microglia Through Targeting CD33 with Liposomes Displaying Glycan Ligands

In this study liposomes displaying a high-affinity CD33 ligand (CD33L) were developed and implemented for engaging CD33. The term “CD33L” is used in this Example to reference the specific CD33 ligand displaying liposomes used in this study, however, this is not intended to restrict the CD33L referenced above to liposomes displaying only this high-affinity ligand; other high-affinity ligands can be used as described above.

The results show that CD33L liposomes enhance phagocytosis in a CD33-dependent manner. CD33L liposomes were formulated to be suitable for investigating the mechanism by which they modulate immune cells and are based on an FDA-approved formulation for use in vivo. Tracking expression of CD33 on the cell surface and entry of liposomes into the cells, this study demonstrates that the ability of the CD33L liposomes to enhance phagocytosis strongly correlates with CD33 internalization. Moreover, CD33L liposomes enhance phagocytosis of primary mouse microglia expressing CD33 following an intracerebroventricular injection of the CD33L liposomes. These results demonstrate the pharmacological value of modulating immune cell function in the brain through targeting CD33, an immunomodulatory glycan-binding protein.

### Methods

Reagents and instrumentation. Reagents were purchased from commercial sources as noted and used without additional purification. NHS-pHrodo, anhydrous DMF, and Et₃N were purchased from Sigma-Aldrich, Canada. DSPE-PEG-NH₂ (2000) and NHS-PEG DSPE were purchased from Avanti. CDCl₃ and MeOD₄ were purchased from Deutero GmbH. Other solvents (analytical and HPLC grade) and reagents were purchased from Aldrich and were used as received. Reactions were monitored by analytical TLC on silica gel 60-F254 (0.25 nm, Silicycle, QC, Canada). Developed TLC plates were visualized under UV lamp (λ max=254 nm) and charred by heating plates that were dipped in ninhydrin solution in ethanol, and acidified anisaldehyde solution in ethanol. The reaction mixture was purified by silica gel column chromatography (230-400 mesh, Silicyle, QC, Canada), Sephadex™ G-100 gel filtration chromatography using CH₂Cl₂/MeOH or H₂O as the elution solvent. NMR experiments were conducted on a Varian 600, or 700 MHz instruments in the Chemistry NMR Facility, University of Alberta. Chemical shifts are reported relative to the deuterated solvent peak or 3-(trimethylsilyl)-propionic-2,2,3,3-d 4 acid sodium salt as an internal standard and are in parts per million (ppm). Coupling constants (J) are reported in Hz and apparent multiplicities were described in standard abbreviations as singlet (s), doublet (d), doublet of doublets (dd), triplet (t), broad singlet (bs), or multiplet (m).

Amine coupling to NHS-PEG-DSPE. A mixture of glycan amine (1.25 equiv.) and NHS-activated PEG-DSPE (1 equiv.) were dissolved in anhydrous DMSO (150 μL, ˜10 mM) and placed in a 1.5 mL centrifuge tube at room temperature. The reaction mixture was degassed under N₂ atmosphere. A diluted solution of Et₃N (1.50 equiv.) in dry DMSO was added carefully to adjust pH of the solution ˜7.5-8.0 and the reaction mixture was incubated at room temperature for around 6h. An aliquot of the reaction mixture was taken for TLC (CHCl₃:MeOH:H₂O=˜75:23:2) analysis. Consumption of the NHS-activated PEG-DSPE and progress of conjugation was monitored by TLC in iodine chamber (to stain glycan-PEG-DSPE conjugate and NHS-PEG-DSPE), ninhydrin (to stain glycan amine), and p-anisaldehyde (glycan-PEG-DSPE conjugate) staining solution. The coupling was performed under the extreme anhydrous condition to avoid hydrolysis of the NHS-activated PEG-DSPE. The crude product was loaded to Sephadex™ G-100 gel filtration column using H₂O and the crude product was purified using H₂O as an eluent to afford glycan-PEG-DSPE conjugate as a cotton white powder after lyophilization of fractions having the desired product. The formation of the desired conjugate was confirmed by analysis of 1H NMR and MALDI-TOF-MS spectroscopic analysis.

### Synthesis of CD33L-PEG-DSPE (conjugate 3): Chemical reaction scheme is presented in Scheme 1

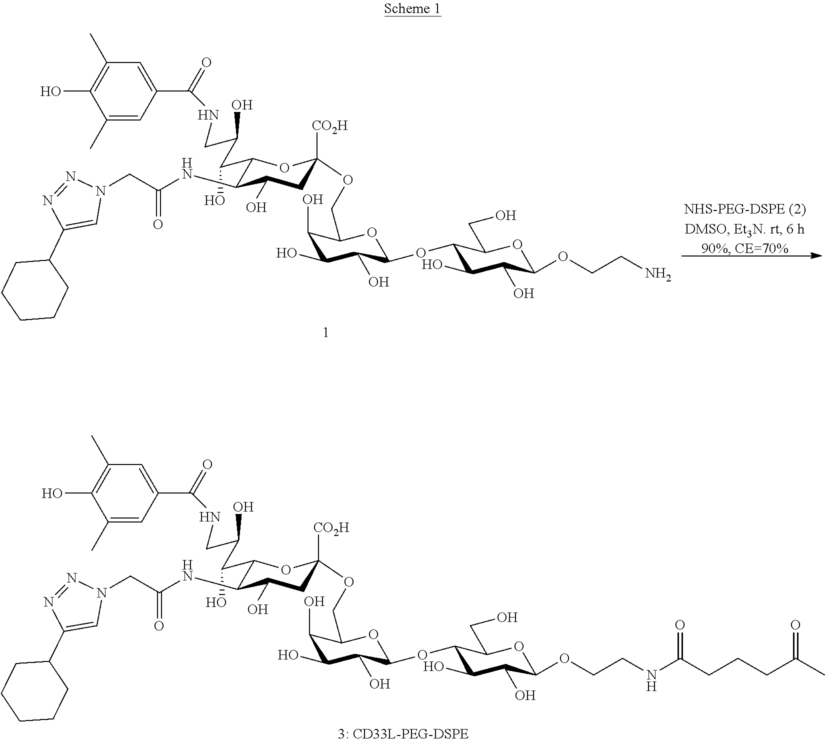

Briefly, conjugate 3 was obtained through coupling of CD33L-NH₂ 1 (1.86 mg, 1.9 μmol, 1.0 equiv) with NHS-activated DSPE-PEG 2 (5 mg, 1.66 μmol, 1 equiv) following the general procedure for conjugation of amine coupling. Yield: (5.81 mg, 90%), coupling efficiency 70%. Coupling efficiency of compound 1 with the DSPE-PEG scaffold was determined by 1H NMR integration. 1H NMR (700 MHz, MeOD₄): δ 7.69 (s, 1H), 7.84 (s, 2H), 5.23-5.20 (m, 1H), 5.09 (dd, J=16.1, 48.6 Hz, 2H), 4.40 (dd, J=3.6, 12.6 Hz, 1H), 4.31 (d, J=7.7 HZ, 1H), 4.29 (d, J=7.7 HZ, 1H), 4.19 (d, J=6.3 HZ, 1H), 4.17-4.15 (m, 2H), 4.06-4.02 (m, 1H), 3.97 (t, J=5.6 Hz, 2H), 3.92-3.86 (m, 5H), 3.82-3.80 (m, 3H), 3.73 (t, J=7.7 HZ, 1H), 3.70-3.62 (m, 5H), 3.64 (broad s, 161H), 3.59-3.51 (m, 12H), 3.48-3.46 (m, 1H), 3.42-3.40 (m, 4H), 3.34-3.32 (m, 3H), 3.31 (broad s, 44H), 3.24 (t, J=6.3 Hz, 2H), 3.04 (t, J=6.3 Hz, 1H), 2.34-2.29 (m, 5H), 2.23 (s, 6H), 2.22-2.18 (m, 4H), 2.42-2.22 (m, 2H), 1.89 (t, J=6.8 Hz, 1H), 1.84-1.72 (m, 2H), 1.76-1.73 (m, 2H), 1.60-1.58 (m, 4H), 1.44-1.42 (m, 4H), 1.30 (broad s, 64H), 0.89 (s, 6H); The MALDI-TOF-MS spectrum showed the average mass centered at 3.8 kDa and expected average mass was 3.8 kDa.

### Synthesis of pHrodo-PEG-DSPE (conjugate 6). Chemical Reaction Scheme is Presented in Scheme 2

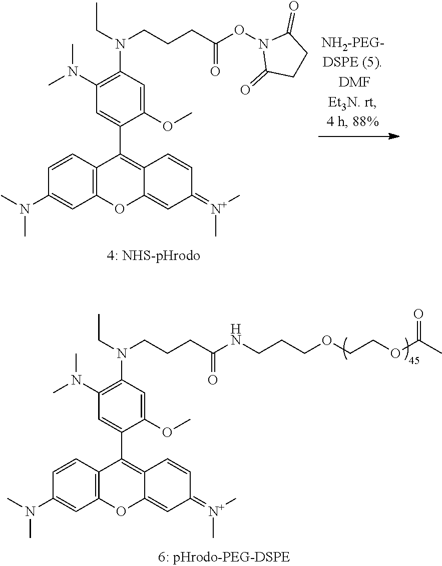

To a solution of NHS-activated pHrodo 4 (1 mg, 1.52 μmol, 1.0 equiv.) and NH₂—PEG-DSPE 5 (5.3 mg, 1.90 μmol, 1.25 equiv.) in anhydrous DMF (10 mM) was added Et₃N to adjust pH of the reaction mixture between 7.5-8 and the solution was stirred for about 2 h at room temperature under dark. Solvent was removed under reduced pressure and the crude product was purified by flash silica gel chromatography using gradient elution (MeOH to CH₂Cl₂ 1:9 v/v). Yield: (4.90 mg, 88%). Coupling efficiency of compound 6 with the DSPE-PEG scaffold was determined by ¹H NMR integration. ¹H NMR (600 MHz, CDCl₃): δ 7.54 (d,J=9.6 Hz, 2H), 7.45 (s, 1H), 7.10 (s, 1H), 7.01 (d, J=1.8 Hz, 1H), 6.99 (d, J=1.8 Hz, 1H), 6.80 (d, J=1.8 Hz, 1H), 6.67 (d, J=28.2 Hz, 1H), 5.24-5.22 (m, 1H), 4.42-4.22 (m, 1H), 4.20-4.18 (m, 2H), 3.98-3.99 (m, 2H), 3.76-3.68 (m, 2H), 3.64 (broad s, 182H), 3.54-3.51 (m, 2H), 3.48-3.44 (m, 4H), 3.42-3.40 (m, 2H), 3.34 (s, 6H), 2.70 (s, 3H), 2.50 (t, J=6.0 Hz, 1H), 2.31-2.27 (m, 3H), 2.05-2.03 (m, 2H), 1.93-1.91 (m, 2H), 1.27 (broad s, 64H), 1.15 (t, J=6.6 Hz, 3H), 0.89 (s, 6H), 0.85 (s, 6H); The MALDI-TOF-MS spectrum showed the average mass centered at 3680 Da and expected average mass was 3680 Da.

Liposome Preparation. Commercially available lipids such as DSPC, Cholesterol, DSPE-PEG were suspended in chloroform and an appropriate volume of each lipid solution in chloroform was transferred into a glass test tube to reach the desired mol % of each lipid. The solvent was removed under nitrogen gas to form the lipid mixtures. Once all visible chloroform was removed approximately 100 μL of dimethyl sulfoxide (DMSO) was added to the test tube. Glycan ligands (CD33L) and 0.1% of DSPE-PEG-A647 in DMSO were then added to the lipid mixture in appropriate proportions to reach the desired mol % of each CD33L. The samples were then placed at −80° C. until completely frozen and excess DMSO was removed via lyophilization overnight and then the dried liposomes were stored at −80° C. until they were extruded.

Dried lipids were then allowed to warm to room temperature and hydrated with 1.0 mL of phosphate-buffered saline pH 7.4 (Gibco). The hydrated lipids were then sonicated in a cycle of 1 minute on, 4-5 minutes off until all lipids were uniformly suspended. The lipids were then extruded with an 800 nm filter and then 100 nm filters. The size of the liposomes was verified by dynamic light scattering (Malvern PanalyticalZetasizer Nano S™) to be approximately 110 nm. Liposomes were stored in a 4° C.

AnimalStudies. All mice were on a C57BL/6J genetic background. Transgenic mice expressing hCD33M in the Rosa26 locus were prepared according to previously published methodology.²⁶ These mice are widely characterized and tested as reported in recently published studies.^{7,8} All animals used were maintained in an access-controlled barrier facility under specific-pathogen-free conditions. Studies were performed in accordance with Public Health Service guidelines and approved (AUP00002885) by the Animal Subjects Committee of the University of Alberta.

Cell lines. Commercially purchased WT U937 (ATCC: CRL-1593.2) cells were used for most of the studies. To generate CD33^{-/-}cells, CRISPR/Cas9-mediated deletion of CD33 in U937 was performed similarly as described in previously published articles.^{7,8} According to these methodologies,^{7,8} site-directed mutagenesis of the intracellular signaling residues (R119A, ITIM, and ITIM-like motifs) also was carried out to achieve mutated versions of CD33.

Liposome binding studies. U937 cells were grown to a density of ˜1×10⁶ cells/mL in a T175 flask before the assay, harvested, centrifuged, resuspended in media, and 100,000 cells were added to a 96-well U-bottom plate in 200 μL of media. To initiate the assay, cells were centrifuged at 300 g for 5 min and the supernatant was discarded. The cell pellet was re-suspended in 50 μL of fresh media and 50 μL of media containing liposomes was added to it. The final concentration of liposomes was 100 μM in each well. The CD33 ligand concentration on the liposomes was 3.33% and the liposome size was 100 nm. These suspensions were incubated for 60 minutes at 37° C. and 4° C. (as negative control). Following this incubation, 100 μL of media was added to each sample and they were centrifuged at 300 g for 5 min. After centrifugation, the supernatant was discarded, and the cell pellet was suspended in flow buffer and further analyzed by flow cytometry. All synthesized liposomes were fluorescently labeled with Alexa 647 fluorophore, which allows us to determine the target specificity of synthesized liposomes to engage CD33 receptor on the cell surface. The extent of this parameter was determined by assessing the median fluorescence intensity (MFI) of the Alexa 647 fluorescent signal. In each case, cells without liposome treatment were kept to subtract non-specific signals from experimental values.

Optimization of liposome formulations and their effect on CD33 internalization. U937 cells were grown in a T175 flask and 100,000 cells were added to a 96-well U-bottom plate in 200 μL of media. To start the assay, cells were centrifuged at 300 g for 5 min and cell-pellet was re-suspended in 50 μL of fresh media, and 50 μL of media containing liposome was added to it. These suspensions were incubated for 120 minutes at 37° C. and 4° C. To best optimize the assay we started with liposome containing different concentrations of ligand (0.1%, 0.33%, 1%, and 3.33%). Furthermore, we varied liposome concentration (1, 10, 100, or 200 μM) to understand how this factor can influence the internalization. Finally, we performed a time (10, 30, 60, 120 240, and 360 min) depended-internalization assay with synthesized liposomes. Following the incubation period with the desired liposome, 150 μL of flow buffer was added in each well, and samples were centrifuged at 300 g for 5 min. After centrifugation supernatant was discarded and the pellet was stained with 50 μL FITC (fluorescein isothiocyanate) labeled anti-human CD 33 antibody (HIM3-4 antibody; 1:100 dilution from a 1 mg/mL stock solution) for 30 minutes at 4° C. Following this incubation 150 μL of flow buffer was added to each sample and they were centrifuged at 300 g for 5 min. After centrifugation, the supernatant was discarded, and the cell pellet was suspended in flow buffer for further analysis by flow cytometry. The extent of decrease of CD33 from cell surface was determined by assessing the MFI of the fluorescent signal observed in FITC channel and using the following formula: Decrease of CD33 from the cell surface (%)=100×(Cells treated with liposome at 37° C.−Cell without liposome treatment 37° C.)/(Cells treated with liposome at 4° C.−Cell without liposome treatment 4° C.)

The addition of pHrodo and Alexa 647 fluorophores to the prepared liposomes, allowed determination of cellular internalization and binding capability of liposomes, respectively. The extent of these two parameters was determined by assessing the MFI of the pHrodo (PE channel) and Alexa 647 fluorescent signal respectively. In each case cells without liposome treatment were retained to determine non-specific signals and treatment at 4° C. was employed as a negative control.

Effect of liposome on cellular phagocytosis. U937 cells were grown in a T175 flask and 100,000 cells were added to a 96-well U-bottom plate in 200 μL of media. To initiate the assay, cells were centrifuged at 300 g for 5 min and the supernatant was discarded. The cell pellet was re-suspended in 50 μL of fresh media and 50 μL of media containing liposomes was added to it. The final concentration of liposomes was 100 μM in each well. The CD33 ligand concentration on the liposomes was 3.33% and the liposome size was 100 nm. In parallel cells were treated with naked liposome which did not contain CD33 ligand. These suspensions were incubated for 120 minutes at 37° C. or 4° C. (negative control). Following the incubation period, 100 μL of media was added to each well, and samples were centrifuged at 300 g for 5 min. After centrifugation, the supernatant was discarded. The cell pellet was re-suspended in 50 μL of fresh media and 50 μL of fluorescent polystyrene beads for 30 minutes at 37° C. or 4° C. The final concentration of the polystyrene beads in each well was a 1:100 dilution from a commercially purchased stock solution. Following this incubation, 100 μL of media was added to each sample and they were centrifuged at 300 g for 5 minutes. After centrifugation, the supernatant was discarded, and the pellet was suspended in flow buffer for further flow cytometric analysis. The extent of phagocytosis was determined by assessing the percentage of cells taking up at least one bead.

CD33L-PEG-DSPE insertion into cells. To initiate the assay, cells were centrifuged at 300 rcf for 5 minutes and the media was discarded. The cells were resuspended in 50 μL of PBS containing 10 μM CD33L-PEG-DSPE or PEG-DSPE and incubated for 1 hr at 37° C. Cells were centrifuged at 300 rcf for 5 min and the supernatant was discarded. Cells were used in either a phagocytosis assay with polystyrene beads or stained with CD33-Fc according to a published procedure²⁰.

Treatment of primary mouse microglia with CD33L liposome. Adult mice (WT and hCD33M) were euthanized under CO₂ and their brain was collected and kept in ice-cold RPMI media with 10% FBS, 100 U/mL Penicillin, and 100 μg/ml Streptomycin. Isolated brains were homogenized by 5 ml syringe plungers in media through 40 am corning filter units under sterile conditions. Homogenized samples were centrifuged at 500 g for 5 min and the pellet was treated with 3 ml of red blood cell lysis buffer (150 mM NH₄Cl, 9 mM NaHCO₃, and 0.1 mM EDTA). Following centrifugation at 300 g for 5 min, the pellet was dissolved in 3 ml of 30% Percoll (Percoll PLUS™, GE Healthcare) and carefully layered on top of 70% Percoll and immediately centrifuged (650 g for 20 min). Immune cells were isolated from the border between the two layers, washed (300 g, 5 min), and resuspended in media. Isolated microglia from WT and hCD33M mice were mixed and treated with naked liposome or CD33L liposome (100 μM) containing 3.33% ligand for 60 min at 37° C. After the incubation period 100 μL fresh media was used to wash excess liposomes and cells were centrifuged at 300 g for 5 min. Pellet was collected and treated with 200 nM aggregated Aβ or polystyrene beads (1:200 dilution from commercial stock) at 37° C. for 30 min. Following this incubation, 100 μL of media was added to each sample and they were centrifuged at 300 g for 5 minutes. After centrifugation, the supernatant was discarded, and the pellet was treated with an antibody cocktail containing CD11b (APC/Cy7, clone M1/70, BioLegend), Ly-6G (BV605, clone 1A8, BioLegend), Ly-6C (BV711, clone HK 1.4, BioLegend), Cx3cr1 (PerCP/Cy5.5, clone SA011F11, BioLegend), and F4/80 (BUV395 clone T45-2342, BD Horizon), hCD33 (APC) at 4° C. for 30 min. After the antibody staining, 50 μL flow buffer was added to the cell pellet and centrifuged at 300 g for 5 minutes. After centrifugation the cell pellet was re-suspended in flow buffer and further analyzed by flow cytometry. To inhibit phagocytosis, cells were pre-treated with 10 μM Cytochalasin-D for 30 min.

Effect of CD33L liposome in CD33 mutant U937 cell lines. WT and mutant U937 cells were grown in a T175 flask and 100,000 cells were added to a 96-well U-bottom plate in 200 μL of media. Phagocytosis assay was performed with polystyrene beads following the method described earlier. The extent of phagocytosis was determined by assessing the percentage of cells taking up at least one bead. A separate assay was performed to also test depletion of cell surface CD33 by FITC-tagged HIM 3-4 antibody, following the method described earlier.

In vivo administration of CD33L liposomes via intracerebroventricular (ICV) injection. WT and CD33 transgenic animals were anesthetized with isoflurane and immobilized on a stereotaxic device. Stereotaxic marking of the lateral ventricles was performed. Briefly, ICV injections of 1 μl CD33L liposome (20 mM) or naked liposome (20 mM) were performed in lateral ventricles with pre-optimized coordinates to ensure successful injection inside the left ventricle. After injection animals were kept in a recovery incubator for 4 hr and then they were euthanized under CO₂. Furthermore, their brain samples were collected and primary microglia were isolated according to the methodology described above. After isolation of microglia from both WT and CD33 transgenic mice, a competitive phagocytosis assay was performed with 200 nM fluorescently labeled aggregated Aβ for 30 min at 37° C. After phagocytosis cells were stained with an antibody cocktail and the extent of phagocytosis was measured by flow cytometric methods as described above.

### Results

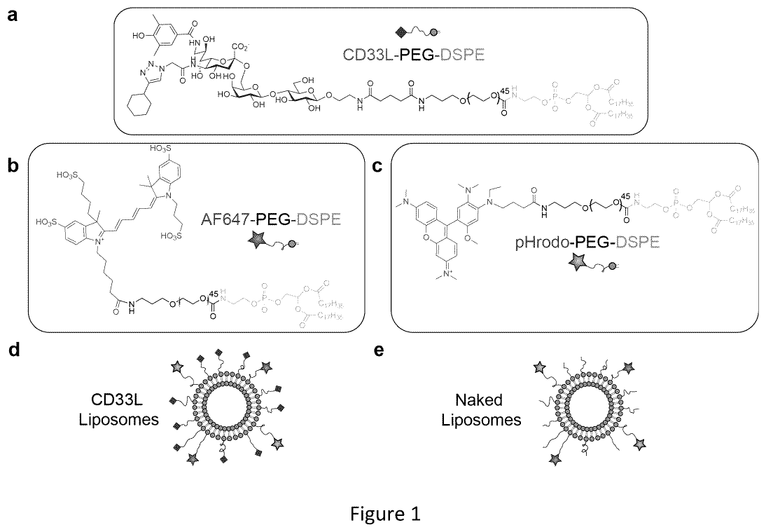

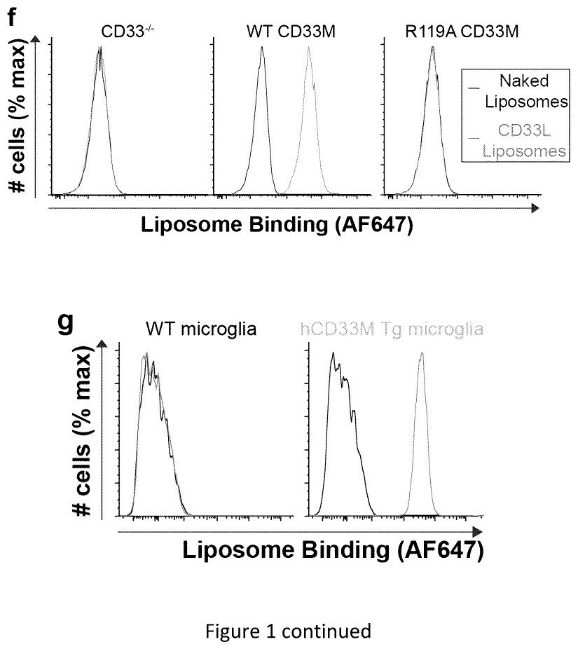

Targeting CD33 with CD33L liposomes. A bifunctionally-modified version of Neu5Acα2-6 lactose that has both selectivity²³ and increased affinity (K_{d}=87 μM)²⁰ for CD33 was used in these studies. For incorporation into liposomal nanoparticles, this synthetic CD33 ligand (CD33L) was conjugated to PEGylated distearoylphosphatidylethanolamine (PEG-DSPE) to form CD33L-PEG-DSPE (FIG. 1a). For monitoring binding and internalization of liposomes to CD33-expressing cells, two fluorophores—AlexaFluor647™ (AF647) and pH-sensitive rhodamine (pHrodo™), respectively—were also conjugated to lipid for incorporation into liposomes (FIG. 1b, c). Using these lipid conjugates, liposomal nanoparticles were initially formulated to contain 0.1 mol % AF647-PEG-DSPE, 0.1 mol % pHrodo-PEG-DSPE, and with (CD33L lipsomes; FIG. 1d) or without (naked liposomes; FIG. 1e) 3 mol % CD33L-PEG-DSPE. The liposome formulation was modelled on an FDA-approved formulation that is well tolerated in vivo.²⁵⁻²⁷ Specifically, the base formulation of these liposomes contained a total of 5 mol % PEGylated lipids, along with 57% distearoylphosphatidylcholine, 38% cholesterol, and were extruded to 100 nm in size as determined by dynamic light scattering.

Liposomes were initially tested for binding to U937 cells by flow cytometry using signal in the AF647 channel as a measure of total binding. CD33L liposomes showed robust binding to WT U937 cells, with no binding to CD33^{-/-} U937 cells or CD33^{-/-} U937 cells transduced with an R119A mutant of CD33 (FIG. 1f). Likewise, CD33L liposomes bound strongly to primary mouse microglia from human CD33M transgenic mice but not WT mouse microglia (FIG. 1g). These results demonstrate that CD33L liposomes specifically engage hCD33M on the cell surface of immune cells, which is in line with previous observations using a multivalent display of this ligand.^{23,26}

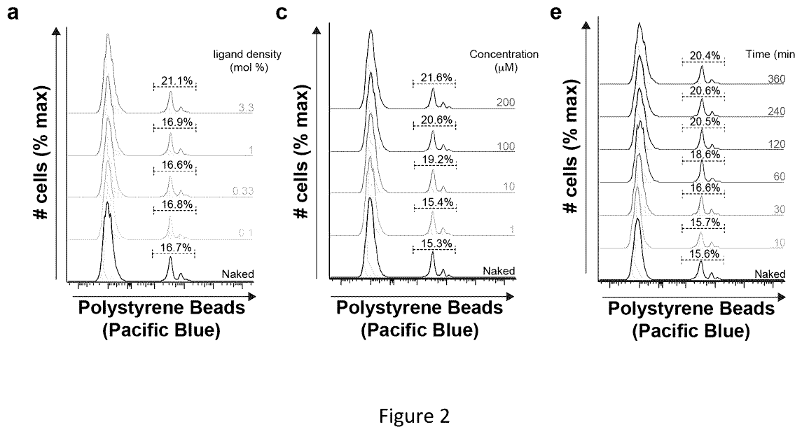

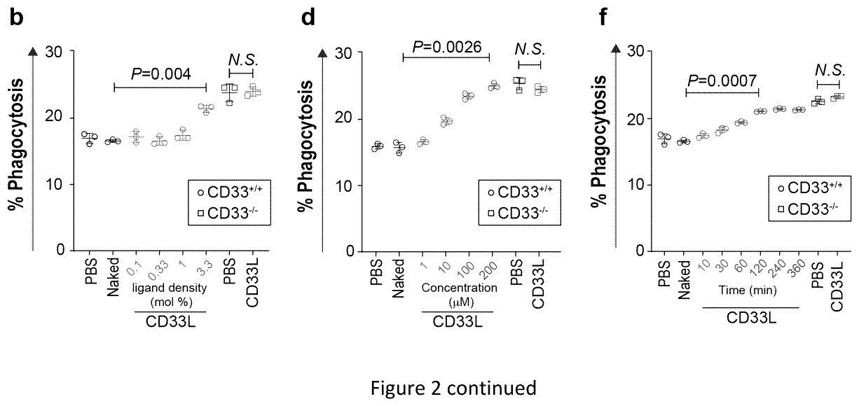

CD33L liposomes increase phagocytosis in a CD33-dependent manner. The effect of engaging CD33 with CD33L liposomes on phagocytosis was examined by incubating cells with liposomes, washing the cells, and monitoring phagocytosis using fluorescent carboxylate-modified polystyrene beads in conjunction with flow cytometry. Initial experiments with the standard formulation (3.3% CD33L liposomes) significantly enhanced phagocytosis in WT but not CD33^{-/-} U937 cells. Therefore, a series of CD33L liposomes were prepared with decreasing amounts of CD33L (FIG. 2a,b). Under these conditions, the effects on phagocytosis were almost lost at CD33L densities below 1 mol %. The further studies were made using with 3.3 mol % CD33L density for further optimizations. Varying the concentration of CD33L liposomes, it was found that enhanced phagocytosis strongly corrected with the concentration of CD33L liposomes used and, notably, the highest concentration increased phagocytosis of WT U937 cells to nearly the level of CD33^{-/-} cells (FIG. 2c,d). As a final optimization 3.3% CD33L liposomes at a concentration of 100 μM were incubated for different amounts of time, with results indicating that phagocytosis increased with incubation up to 2 hr, with no further increase was observed beyond 2 hr (FIG. 2e,f). These data indicate that engagement of CD33 by CD33L liposomes increases phagocytosis in U937 cells in a manner that is dependent on CD33L density, liposome concentration, and time with which the liposomes are incubated with cells.

Recently, the present inventors and others have demonstrated that the CD33 long isoform (CD33M) represses microglial phagocytosis, which is consistent with CD33M acting as an immunoinhibitory receptor.^{7,10} These results suggest that engaging CD33 with CD33L liposomes reverses this inhibitory effect. While surprising, this is conceptually similar to results observed for another member of the Siglecs, CD22, where anti-CD22 antibodies enhance microglial phagocytosis.²⁸

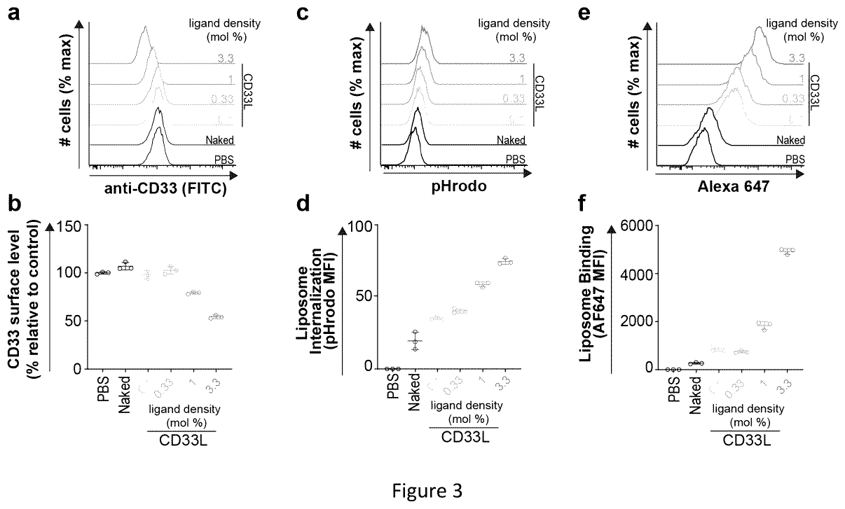

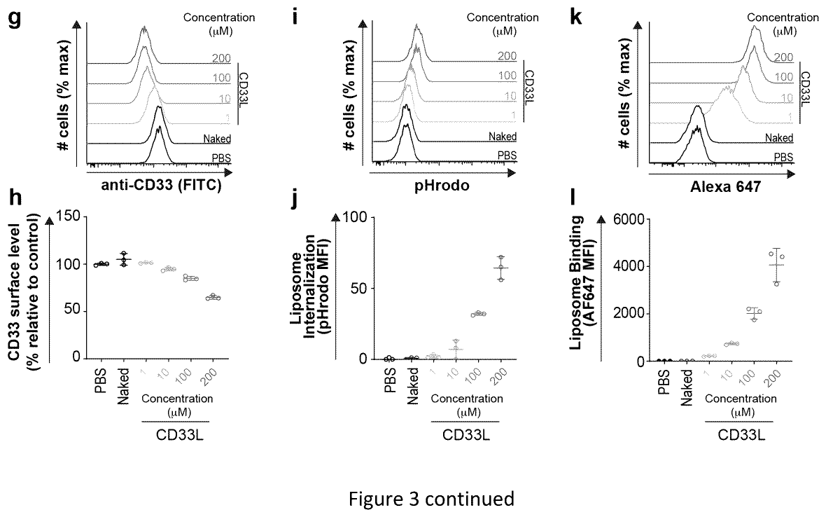

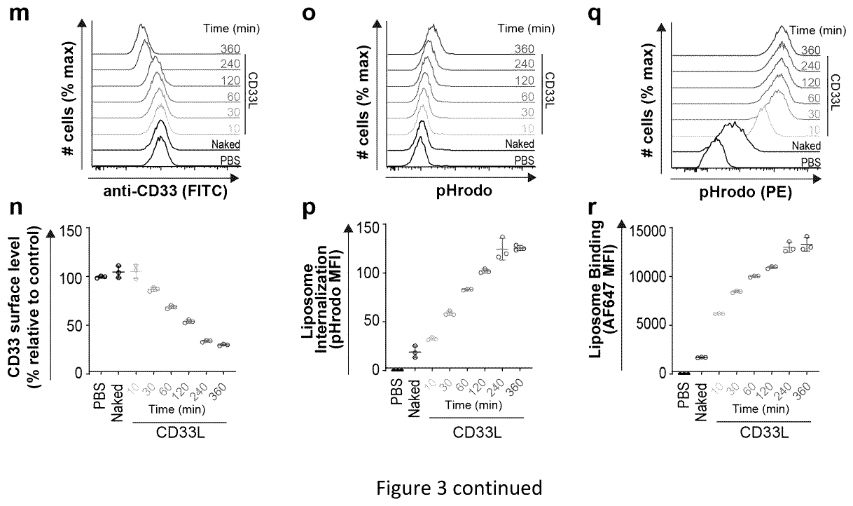

CD33L liposomes depletion cell surface CD33. As Siglecs are endocytic receptors,²⁹ CD33L liposomes were studied to show they induce internalization of CD33. Internalization was assessed by monitoring the depletion of CD33 from the cell surface using cell surface staining with an anti-CD33 antibody. A specific antibody clone (HIM3-4)³⁰ was chosen that does not bind the glycan-binding domain of CD33 to ensure minimal interference from CD33L liposomes. In parallel, internalization of the liposomes was assessed using the fluorescent signal from pHrodo™ incorporated into the liposomes and comparing it to the AF647 signal that represents total liposome binding. For liposomes where the CD33L density was varied, 3.3 mol % CD33L induced the most significant depletion of CD33 from the cell surface (50% depletion), liposome internalization, and liposome binding (FIG. 3a-f). Liposomes bearing 1 mol % CD33L produced a modest depletion of cell surface CD33 levels as well as CD33 internalization, yet did not significantly increase phagocytosis (FIG. 2a,b). Examining the concentration-(FIG. 3g-l) and time-dependent (FIG. 3m-r) effects of CD33L liposomes on internalization revealed that internalization of both CD33 and the CD33L liposomes strongly correlate with enhanced phagocytosis. Specifically, 200 μM liposomes led to the greatest internalization of CD33 and liposomes, while internalization plateaued at 2 hr.

Previous work using 5 μM beads displaying the same ligand used in the present study also induced an increase in phagocytosis, but due to the very large size of these beads it is unknown if they can be internalized and whether depletion of cell surface CD33 occurred.²⁴ In contrast, the present results demonstrate for the first time that internalization of CD33 closely correlates with increases in phagocytosis induced by CD33L liposomes. Interestingly, total CD33 engagement did not necessarily correlate with internalization and, consequently, increased phagocytosis. For example, the CD33L liposomes bearing 1 mol % of the ligand still bound to CD33-expressing cells as evidenced by the AF647 signal (FIG. 3e), but did not show significant depletion of CD33 from the cell surface (FIG. 3a) or increased phagocytosis (FIG. 2a). Contrasting with this result are 10 μM of liposomes (with the optimal 3.3 mol % CD33L), which had a similar overall ability to bind to cells (FIG. 3k) but did deplete cell surface CD33 levels (FIG. 3g) and increase phagocytosis (FIG. 2c). Without wishing to be bound by theory, it is hypothesized that the degrees of crosslinking of CD33 may be an important factor in inducing its internalization. Demonstrating that removal of CD33 from the cell surface—the location where it plays its immunomodulatory function—is directly responsible for the increased phagocytosis would require conditions where CD33 cannot internalize. However, compounds that block endocytosis would necessarily also impact phagocytosis.

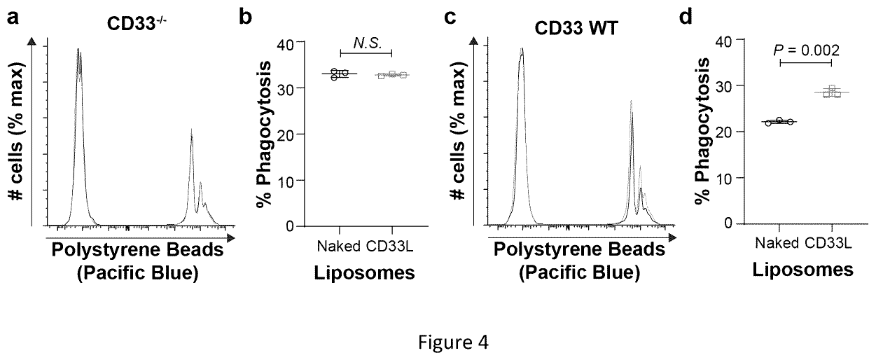

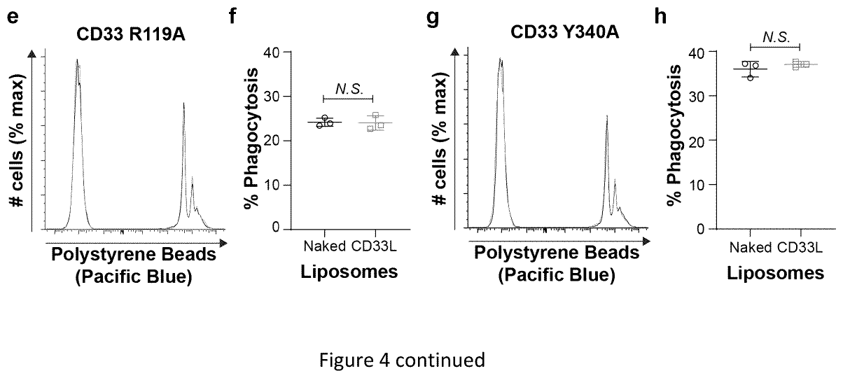

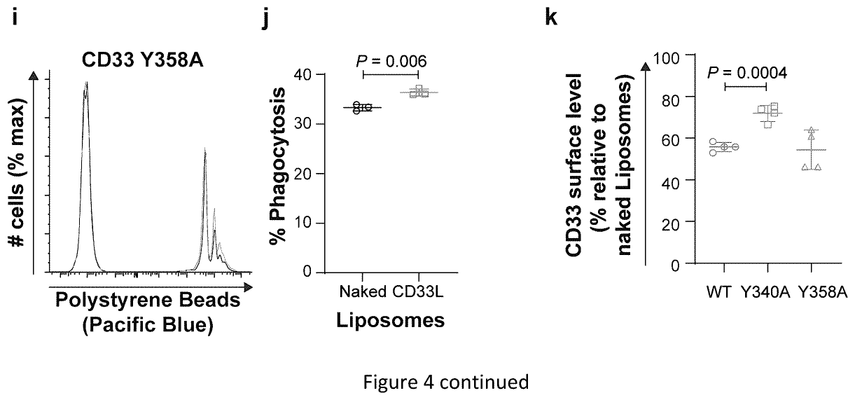

Role of glycan-binding and signaling motifs on phagocytosis. Binding of CD33 to its glycan ligands is dependent on a key salt bridge formed between Arg119 within its N-terminal V-set domain²⁴, while signal motifs are contained within its cytoplasmic tail at Tyr340 (in the immunoreceptor tyrosine-based inhibition motif (ITIM)) and Tyr358 (ITIM-like)³¹. CD33^{-/-} U937 cells were previously established with WT CD33, R119A CD33, Y340A CD33, and Y358A CD33 re-introduced in these cells through lentiviral transduction⁸. Here, these mutants were used to interrogate the roles of these key residues in the enhancement of phagocytosis by CD33L liposomes. Cell lines were treated with 100 μM CD33L liposomes (3.3% CD33L) for 60 min prior to carrying out a phagocytosis assay with polystyrene beads. Compared to CD33^{-/-} cells (FIG. 4a,b), cells reconstituted with WT CD33 showed enhanced phagocytosis by the treatment of CD33L liposomes (FIG. 4c,d). In both the R119A (FIG. 4e,f) and Y340A (FIG. 4g,h) mutants, the phagocytosis-enhancing effect of CD33L liposomes was lost. CD33L liposomes were still capable of enhanced phagocytosis in cells expressing the Y358A mutant (FIG. 4i,j). Previously, the present inventors have shown that Y340 was critical for the ability of CD33 to repress phagocytosis, however, others have also shown with antibodies that Y340 is required for CD33 endocytosis^{32,13}. Therefore, it was not clear whether a loss in the ability of CD33L liposomes to enhance phagocytosis was due to loss of this critical signaling motif, or the inability of this mutant to internalize. Examining the ability of CD33L liposomes to induce CD33 internalization, it was shown that the Y340A mutant internalizes significantly less CD33 as compared to WT CD33 (FIG. 4K). However, there was no visible difference was observed between WT CD33 and the Y358A mutant. These results establish that the ability of CD33 to bind its glycan ligands and repress signaling is essential for the ability of CD33L liposomes to enhance phagocytosis. Furthermore, it also supports that the ITIM motif is involved in CD33 internalization.

Recently, it was found that the ITIM of CD33 is essential for its ability to repress phagocytosis, whereas the ITIM-like residue is dispensable. The findings here demonstrate that crosslinking alone of CD33, without its ability to modulate immune cell signaling through the ITIM, is insufficient for CD33L liposomes to impact phagocytosis. Previous results demonstrating a requirement for the ITIM of CD33 in antibody-mediated internalization³³ agree with the present findings. The optimized formulation of CD33L liposomes display from about 1000 to about 2000 molecules of CD33L based on a 100 nm liposome containing 84,000 lipids. This high multivalency combined with an optimized size of about 100 nm for clathrin-dependent endocytosis gives liposomes a strong ability to internalize surface receptors.³⁴⁻³⁷

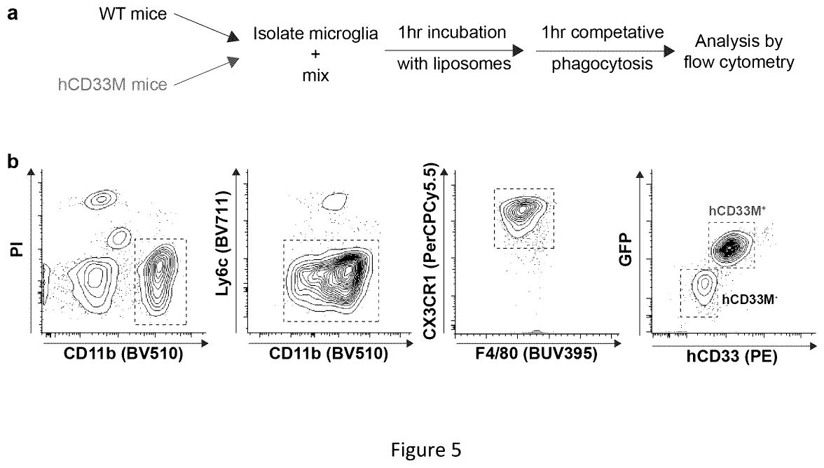

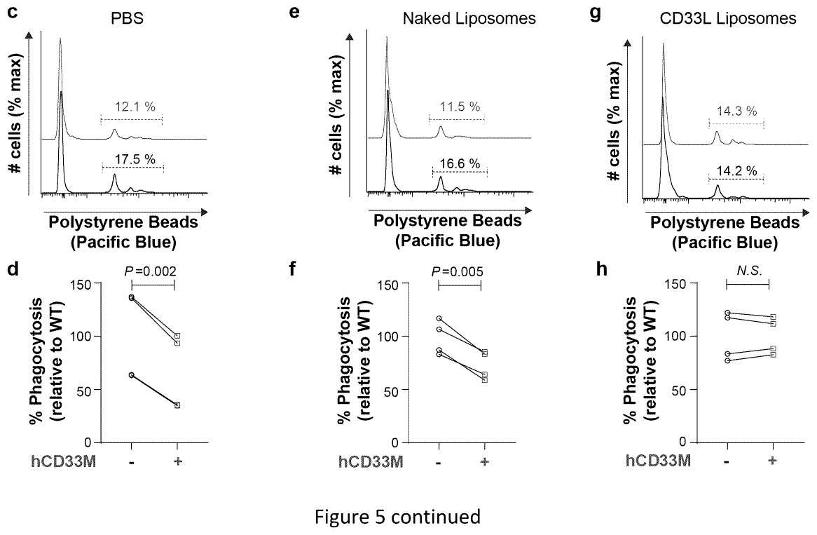

CD33L liposomes increase phagocytosis in primary microglia ex vivo. Results demonstrating that CD33L liposomes enhance phagocytosis in cultured U937 cells prompted the study to demonstrate a similar effect in microglia. To do so, microglia were isolated from WT and hCD33M transgenic mice and a competitive phagocytosis assay was performed following incubation of cells with liposomes (FIG. 5a). Phagocytosis within the hCD33M⁺ and hCD33M⁻ populations were deconvoluted using the gating scheme shown in FIG. 5b. The mixture of cells was incubated with PBS (FIG. 5c,d), naked liposomes (FIG. 5,e,f), or CD33L liposomes (FIG. 5g,h). hCD33M transgenic microglia showed a decrease in phagocytosis compared to WT microglia in conditions where cells were treated with PBS and naked liposomes, which was anticipated based on the ability of hCD33M to repress phagocytosis in these transgenic mice^{7,8}. Pre-treatment with CD33L liposome increased phagocytosis in the CD33M microglia such that a statistically significant difference was observed in phagocytosis between the WT and hCD33M transgenic microglia. These results demonstrate that CD33L liposomes can enhance microglial cell phagocytosis in a CD33-dependent manner.

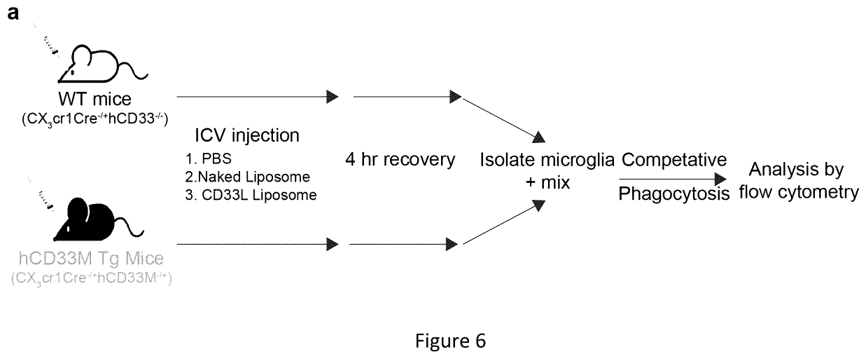

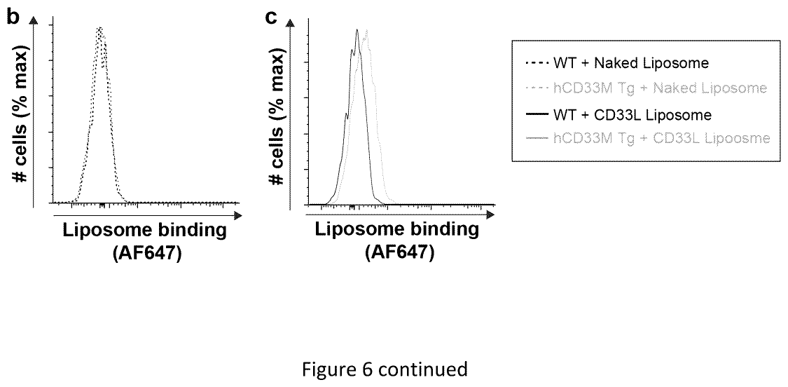

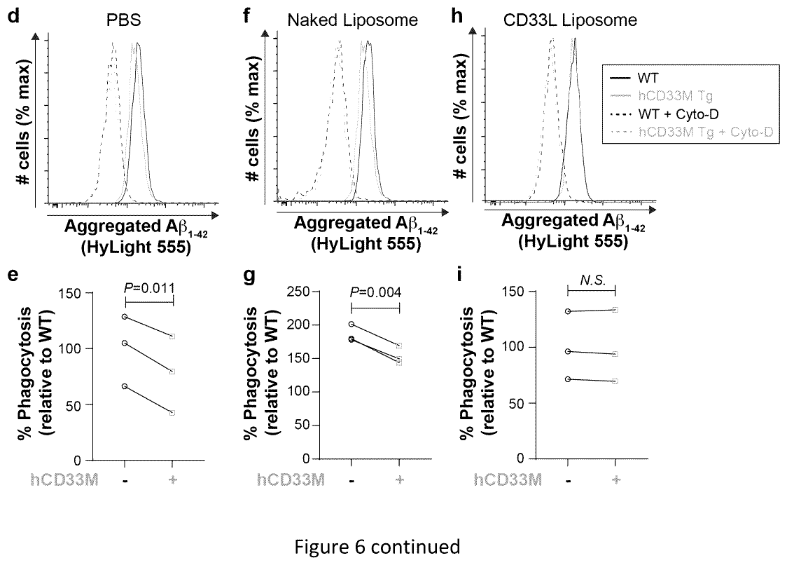

In vivo administration of CD33L liposomes enhanced phagocytosis in microglia. An advantage of the present liposome formulation is that it is based on an FDA-approved formulation that is well-tolerated in vivo.²⁶ Notably, however, this formulation does not penetrate the blood-brain barrier.³⁹ Therefore, to demonstrate CD33L liposomes successfully stimulate microglial phagocytosis in vivo, the liposomes were administered to mice via intracerebroventricular (ICV) injection. Briefly, WT and hCD33M transgenic mice were ICV injected with PBS, naked liposomes, or CD33L liposomes into the left ventricle, followed by a 4 hr recovery prior to euthanization of mice, collection of the brain, and isolation of microglia (FIG. 6a). Initially, a test was performed with AF647 labelled naked and CD33L liposomes and AF647 fluorescence was examined in isolated microglia, which demonstrated successful targeting of hCD33M-expressing cells selectively with CD33L liposomes (FIG. 6b,c). For illustrating the impact on phagocytosis, studies using WT and hCD33M Tg mice were carried out in parallel, and a competitive phagocytosis assay was used. In these studies, phagocytosis was assessed using the more physiologically-relevant aggregated fluorescent Aβ₁₋₄₂ and using conditions with and without Cytochaslin-D to control for non-specific sticking or uptake. Consistent with the results described above, hCD33M microglia mice injected with PBS (FIG. 6d,e) or naked liposome (FIG. 6f,g) showed repressed phagocytosis compared to their WT counterpart. On the other hand, CD33L liposome-injected hCD33M mice (FIG. 6h,i) showed a similar level of phagocytosis as compared to WT mice under identical conditions. These data demonstrate that in vivo administration of CD33L liposome effectively modulates phagocytosis in microglia in a CD33-dependent manner.

In summary, the results of this study demonstrate that CD33L liposomes can increase microglial phagocytosis both in vitro and in vivo. CD33L conjugated to microparticles produced a similar impact on phagocytosis in vitro, but due to their large size these particles would not be suitable for in vivo use.⁴⁰ Use of a biocompatible liposomal formulation⁴¹, enabled CD33L density to be readily and systematically varied and to be used effectively in vivo.

Previously CD33L liposomes were successfully used to dampen allergic response in mice through exploiting the inhibitory function of CD33 on mast cells.²⁶ It has now been surprisingly found that CD33L liposomes are also capable of increasing microglial phagocytosis. Modulating the effects of microglia in the brain is the next frontier in the treatment of neurodegenerative disease. Clinical studies using antibodies directly targeting Aβ plaque accumulation have shown some promise but ultimately have not been successful. Thus, augmenting the ability of immune cells in the brain may be a better approach. Antibody-based clinical trials are underway to do so and while antibodies have long circulatory half-lives, they have a poor ability to cross the blood brain barrier. Multivalent engagement of Siglecs with glycan ligands offer several advantages over antibodies, including differences in cellular trafficking upon engagement and lower immunogenicity.²² CD33 has long been used a target of leukemia for antibody-drug conjugates, due to the expression of CD33 in AML.¹⁶ The long-term safety associated with CD33-directed antibody therapies is well established and results demonstrating that mice reconstituted with CD33^{-/-} human immune cells showed normal immune cell function provides further evidence that CD33-engaging therapies will well be tolerated.⁴²

This Example clearly demonstrates that the CD33L liposomes of the present application effectively increased phagocytosis of cultured monocytic cells and transgenic mouse microglia in a CD33-dependent manner. Enhanced phagocytosis strongly correlated with loss of CD33 from the cell surface and internalization of liposomes, as monitored by a pH-sensitive fluorophore installed on the liposomes. Cells expressing a mutant of CD33 lacking a critical intracellular signaling motif were not affected by CD33L liposomes, despite this mutant still internalizing, demonstrating that CD33L liposomes reverse the inhibitory signal imparted by CD33. These effects were specific to trans engagement of CD33, as cis engagement produced the opposite effect on phagocytosis. Moreover, intracerebroventricular injection of CD33L liposomes into hCD33 transgenic mice enhanced phagocytosis of microglia.

### REFERENCES

(1) Efthymiou, A. G.; Goate, A. M. Late onset Alzheimer's disease genetics implicates microglial pathways in disease risk. Mol Neurodegener 2017, 12 (1), 43.
(2) Sarlus, H.; Heneka, M. T. Microglia in Alzheimer's disease. J Clin Invest 2017, 127 (9), 3240.
(3) Estus, S.; Shaw, B. C.; Devanney, N.; Katsumata, Y.; Press, E. E.; Fardo, D. W. Evaluation of CD33 as a genetic risk factor for Alzheimer's disease. Acta Neuropathol 2019, 138 (2), 187.
(4) Naj, A. C.; Jun, G.; Beecham, G. W.; Wang, L. S.; Vardarajan, B. N.; Buros, J.; Gallins, P. J.; Buxbaum, J. D.; Jarvik, G. P.; Crane, P. K. et al. Common variants at MS4A4/MS4A6E, CD2AP, CD33 and EPHA1 are associated with late-onset Alzheimer's disease. Nat Genet 2011, 43 (5), 436.
(5) Hollingworth, P.; Harold, D.; Sims, R.; Gerrish, A.; Lambert, J. C.; Carrasquillo, M. M.; Abraham, R.; Hamshere, M. L.; Pahwa, J. S.; Moskvina, V. et al. Common variants at ABCA7, MS4A6A/MS4A4E, EPHA1, CD33 and CD2AP are associated with Alzheimer's disease. Nat Genet 2011, 43 (5), 429.
(6) Malik, M.; Simpson, J. F.; Parikh, I.; Wilfred, B. R.; Fardo, D. W.; Nelson, P. T.; Estus, S. CD33 Alzheimer's risk-altering polymorphism, CD33 expression, and exon 2 splicing. J Neurosci 2013, 33 (33), 13320.
(7) Bhattacherjee, A.; Rodrigues, E.; Jung, J.; Luzentales-Simpson, M.; Enterina, J. R.; Galleguillos, D.; St Laurent, C. D.; Nakhaei-Nejad, M.; Fuchsberger, F. F.; Streith, L. et al. Repression of phagocytosis by human CD33 is not conserved with mouse CD33. Commun Biol 2019, 2, 450.
(8) Bhattacherjee, A.; Jung, J.; Zia, S.; Ho, M.; Eskandari-Sedighi, G.; St Laurent, C. D.; McCord, K. A.; Bains, A.; Sidhu, G.; Sarkar, S. et al. The CD33 short isoform is a gain-of-function variant that enhances Abeta1-42 phagocytosis in microglia. Mol Neurodegener 2021, 16 (1), 19.
(9) Ann Butler, C.; Thornton, P.; Charles Brown, G. CD33M inhibits microglial phagocytosis, migration and proliferation, but the Alzheimer's disease-protective variant CD33m stimulates phagocytosis and proliferation, and inhibits adhesion. J Neurochem 2021, DOI:10.1111/jnc.15349 10.1111/jnc.15349.
(10) Wissfeld, J.; Nozaki, I.; Mathews, M.; Raschka, T.; Ebeling, C.; Hornung, V.; Brustle, O.; Neumann, H. Deletion of Alzheimer's disease-associated CD33 results in an inflammatory human microglia phenotype. Glia 2021, 69 (6), 1393.
(11) Price, B. R. Preclinical Targeting of TREM2 for the Treatment of Alzheimer's Disease-type Pathology in a Transgenic Mouse Model. 2019.
(12) Schlepckow, K.; Monroe, K. M.; Kleinberger, G.; Cantuti-Castelvetri, L.; Parhizkar, S.; Xia, D.; Willem, M.; Werner, G.; Pettkus, N.; Brunner, B. et al. Enhancing protective microglial activities with a dual function TREM2 antibody to the stalk region. EMBO Mol Med 2020, 12 (4), e11227.
(13) Fassler, M.; Rappaport, M. S.; Cuno, C. B.; George, J. Engagement of TREM2 by a novel monoclonal antibody induces activation of microglia and improves cognitive function in Alzheimer's disease models. J Neuroinflammation 2021, 18 (1), 19.
(14) Griciuc, A.; Patel, S.; Federico, A. N.; Choi, S. H.; Innes, B. J.; Oram, M. K.; Cereghetti, G.; McGinty, D.; Anselmo, A.; Sadreyev, R. L. et al. TREM2 Acts Downstream of CD33 in Modulating Microglial Pathology in Alzheimer's Disease. Neuron 2019, 103 (5), 820.
(15) Bradshaw, E. M.; Chibnik, L. B.; Keenan, B. T.; Ottoboni, L.; Raj, T.; Tang, A.; Rosenkrantz, L. L.; Imboywa, S.; Lee, M.; Von Korff, A. et al. CD33 Alzheimer's disease locus: altered monocyte function and amyloid biology. Nat Neurosci 2013, 16 (7), 848.
(16) Walter, R. B. Expanding use of CD33-directed immunotherapy. Expert Opin Biol Ther 2020, 20 (9), 955.
(17) Walter, R. B.; Appelbaum, F. R.; Estey, E. H.; Bernstein, I. D. Acute myeloid leukemia stem cells and CD33-targeted immunotherapy. Blood 2012, 119 (26), 6198.
(18) Malik, M.; Chiles, J., 3rd; Xi, H. S.; Medway, C.; Simpson, J.; Potluri, S.; Howard, D.; Liang, Y.; Paumi, C. M.; Mukherjee, S. et al. Genetics of CD33 in Alzheimer's disease and acute myeloid leukemia. Hum Mol Genet 2015, 24 (12), 3557.
(19) Macauley, M. S.; Crocker, P. R.; Paulson, J. C. Siglec-mediated regulation of immune cell function in disease. Nat Rev Immunol 2014, 14 (10), 653.
(20) Rodrigues, E.; Jung, J.; Park, H.; Loo, C.; Soukhtehzari, S.; Kitova, E. N.; Mozaneh, F.; Daskhan, G.; Schmidt, E. N.; Aghanya, V. et al. A versatile soluble siglec scaffold for sensitive and quantitative detection of glycan ligands. Nat Commun 2020, 11 (1), 5091.
(21) Bull, C.; Nason, R.; Sun, L.; Van Coillie, J.; Madriz Sorensen, D.; Moons, S. J.; Yang, Z.; Arbitman, S.; Fernandes, S. M.; Furukawa, S. et al. Probing the binding specificities of human Siglecs by cell-based glycan arrays. Proc Natl Acad Sci USA 2021, 118 (17).
(22) Angata, T.; Nycholat, C. M.; Macauley, M. S. Therapeutic Targeting of Siglecs using Antibody- and Glycan-Based Approaches. Trends Pharmacol Sci 2015, 36 (10), 645.
(23) Rillahan, C. D.; Macauley, M. S.; Schwartz, E.; He, Y.; McBride, R.; Arlian, B. M.; Rangarajan, J.; Fokin, V. V.; Paulson, J. C. Disubstituted Sialic Acid Ligands Targeting Siglecs CD33 and CD22 Associated with Myeloid Leukaemias and B Cell Lymphomas. Chem Sci 2014, 5 (6), 2398.
(24) Miles, L. A.; Hermans, S. J.; Crespi, G. A. N.; Gooi, J. H.; Doughty, L.; Nero, T. L.; Markulic, J.; Ebneth, A.; Wroblowski, B.; Oehlrich, D. et al. Small Molecule Binding to Alzheimer Risk Factor CD33 Promotes Abeta Phagocytosis. iScience 2019, 19, 110.
(25) Knop, K.; Hoogenboom, R.; Fischer, D.; Schubert, U. S. Poly(ethylene glycol) in drug delivery: pros and cons as well as potential alternatives. Angew Chem Int Ed Engl 2010, 49 (36), 6288.
(26) Duan, S.; Koziol-White, C. J.; Jester, W. F., Jr.; Smith, S. A.; Nycholat, C. M.; Macauley, M. S.; Panettieri, R. A., Jr.; Paulson, J. C. CD33 recruitment inhibits IgE-mediated anaphylaxis and desensitizes mast cells to allergen. J Clin Invest 2019, 129 (3), 1387.
(27) Jusu, S. M.; Obayemi, J. D.; Salifu, A. A.; Nwazojie, C. C.; Uzonwanne, V.; Odusanya, O. S.; Soboyejo, W. O. Drug-encapsulated blend of PLGA-PEG microspheres: in vitro and in vivo study of the effects of localized/targeted drug delivery on the treatment of triple-negative breast cancer. Sci Rep 2020, 10 (1), 14188.
(28) Pluvinage, J. V.; Haney, M. S.; Smith, B. A. H.; Sun, J.; Iram, T.; Bonanno, L.; Li, L.; Lee, D. P.; Morgens, D. W.; Yang, A. C. et al. CD22 blockade restores homeostatic microglial phagocytosis in ageing brains. Nature 2019, 568 (7751), 187.
(29) Biedermann, B.; Gil, D.; Bowen, D. T.; Crocker, P. R. Analysis of the CD33-related siglec family reveals that Siglec-9 is an endocytic receptor expressed on subsets of acute myeloid leukemia cells and absent from normal hematopoietic progenitors. Leuk Res 2007, 31 (2), 211.
(30) Perez-Oliva, A. B.; Martinez-Esparza, M.; Vicente-Fernandez, J. J.; Corral-San Miguel, R.; Garcia-Penarrubia, P.; Hernandez-Caselles, T. Epitope mapping, expression and post-translational modifications of two isoforms of CD33 (CD33M and CD33m) on lymphoid and myeloid human cells. Glycobiology 2011, 21 (6), 757.
(31) Paul, S. P.; Taylor, L. S.; Stansbury, E. K.; McVicar, D. W. Myeloid specific human CD33 is an inhibitory receptor with differential ITIM function in recruiting the phosphatases SHP-1 and SHP-2. Blood 2000, 96 (2), 483.
(32) Walter, R. B.; Raden, B. W.; Kamikura, D. M.; Cooper, J. A.; Bernstein, I. D. Influence of CD33 expression levels and ITIM-dependent internalization on gemtuzumab ozogamicin-induced cytotoxicity. Blood 2005, 105 (3), 1295.
(33) Walter, R. B.; Raden, B. W.; Zeng, R.; Hausermann, P.; Bernstein, I. D.; Cooper, J. A. ITIM-dependent endocytosis of CD33-related Siglecs: role of intracellular domain, tyrosine phosphorylation, and the tyrosine phosphatases, Shp1 and Shp2. J Leukoc Biol 2008, 83 (1), 200.
(34) Paoli, E. E.; Ingham, E. S.; Zhang, H.; Mahakian, L. M.; Fite, B. Z.; Gagnon, M. K.; Tam, S.; Kheirolomoom, A.; Cardiff, R. D.; Ferrara, K. W. Accumulation, internalization and therapeutic efficacy of neuropilin-1-targeted liposomes. J Control Release 2014, 178, 108.
(35) Wang, X.; Qiu, Y.; Wang, M.; Zhang, C.; Zhang, T.; Zhou, H.; Zhao, W.; Zhao, W.; Xia, G.; Shao, R. Endocytosis and Organelle Targeting of Nanomedicines in Cancer Therapy. Int J Nanomedicine 2020, 15, 9447.
(36) Kim, B.; Shin, J.; Kiziltepe, T.; Bilgicer, B. Identification of a moderate affinity CD22 binding peptide and in vitro optimization of peptide-targeted nanoparticles for selective uptake by CD22+B-cell malignancies. Nanoscale 2020, 12 (21), 11672.
(37) Macauley, M. S.; Pfrengle, F.; Rademacher, C.; Nycholat, C. M.; Gale, A. J.; von Drygalski, A.; Paulson, J. C. Antigenic liposomes displaying CD22 ligands induce antigen-specific B cell apoptosis. J Clin Invest 2013, 123 (7), 3074.
(38) Delaveris, C. S.; Chiu, S. H.; Riley, N. M.; Bertozzi, C. R. Modulation of immune cell reactivity with cis-binding Siglec agonists. Proc Natl Acad Sci USA 2021, 118 (3).
(39) Vieira, D. B.; Gamarra, L. F. Getting into the brain: liposome-based strategies for effective drug delivery across the blood-brain barrier. Int J Nanomedicine 2016, 11, 5381.
(40) Kohane, D. S. Microparticles and nanoparticles for drug delivery. Biotechnol Bioeng 2007, 96 (2), 203.
(41) De Jong, W. H.; Borm, P. J. Drug delivery and nanoparticles:applications and hazards. Int J Nanomedicine 2008, 3 (2), 133.
(42) Kim, M. Y.; Yu, K. R.; Kenderian, S. S.; Ruella, M.; Chen, S.; Shin, T. H.; Aljanahi, A. A.; Schreeder, D.; Klichinsky, M.; Shestova, O. et al. Genetic Inactivation of CD33 in Hematopoietic Stem Cells to Enable CAR T Cell Immunotherapy for Acute Myeloid Leukemia. Cell 2018, 173 (6), 1439.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A liposomal composition for inducing microglial phagocytosis, said composition comprising a liposome nanoparticle and a glycan ligand for CD33 displayed on the liposome nanoparticle.

2. The liposomal composition for inducing microglial phagocytosis according to claim 1, wherein the glycan ligand is present at an amount of greater than 1 mol %, preferably from 1 mol % to 5 mol %, more preferably from 3 mol % to 5 mol %, or most preferably about 3.3 mol %.

3. The liposomal composition for inducing microglial phagocytosis according to claim 1, wherein CD33 has a binding affinity for the glycan ligand that is at least about 20 times, preferably at least about 25 times, or more preferably at least about 30 times, stronger than the binding affinity of CD33 for its natural sialic acid ligands.

4. The liposomal composition for inducing microglial phagocytosis according to claim 1, wherein the glycan ligand has a dissociation constant (K_{D}), based on monovalent binding of the glycan ligand with CD33, that is less than 120 μM, less than 110 μM, less than 100 μM, or preferably less than 90 μM.

5. The liposomal composition for inducing microglial phagocytosis according to claim 1, wherein the liposomal composition comprises a glycan ligand formed by lipid attachment at the amine of the compound of formula 1

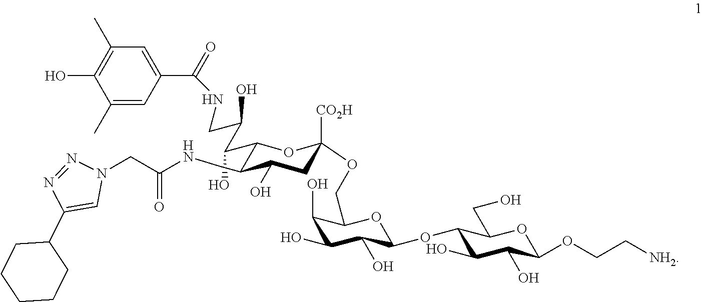

6. The liposomal composition for inducing microglial phagocytosis according to claim 5, wherein the glycan ligand is a compound of formula 3

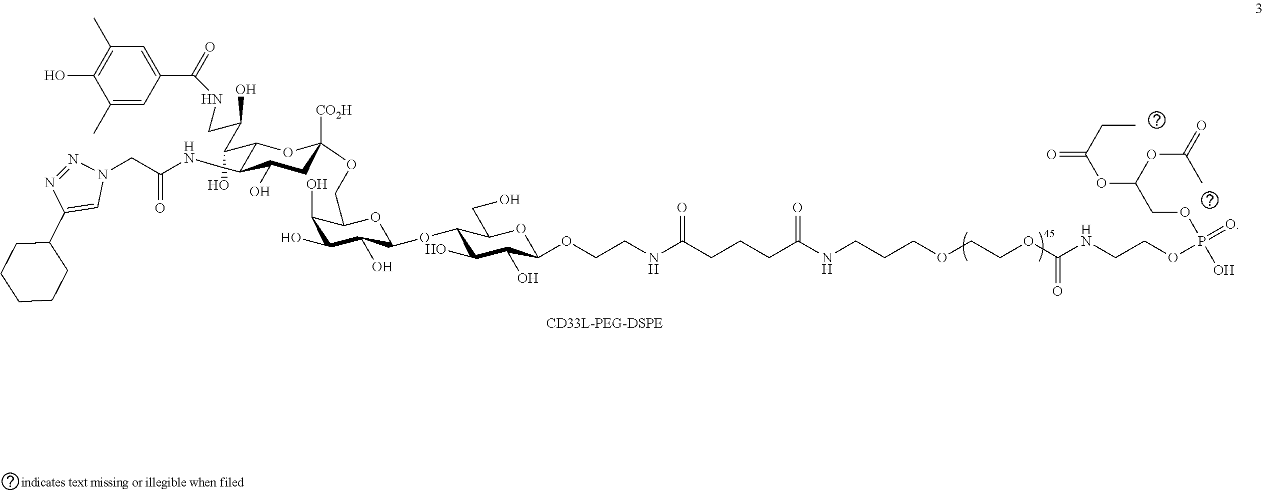

7. The liposomal composition for inducing microglial phagocytosis according to claim 1, wherein the composition is formulated for direct administration to the brain of a subject.

8. The liposomal composition for inducing microglial phagocytosis according to claim 7, wherein the composition is formulated for intracerebroventricular administration to the subject.

9. A method for preventing, treating or delaying the progression of Alzheimer's disease in a subject, said method comprising administering to the subject a liposomal composition comprising a liposome nanoparticle and a glycan ligand for CD33 displayed on the liposome nanoparticle.

10. The method according to claim 9, wherein the liposomal composition is administered in an amount effective to induce microglial phagocytosis in the subject.

11. The method according to claim 9, wherein the glycan ligand is present in the liposomal composition at an amount of greater than 1 mol %, preferably from 1 mol % to 5 mol %, more preferably from 3 mol % to 5 mol %, or most preferably about 3.3 mol %.

12. The method according to claim 9, wherein CD33 has a binding affinity for the glycan ligand that is at least about 20 times, preferably at least about 25 times, or more preferably at least about 30 times, stronger than the binding affinity of CD33 for its natural sialic acid ligand.

13. The method according to claim 9, wherein the glycan ligand has a dissociation constant (K_{D}), based on monovalent binding of the glycan ligand with CD33, that is less than 120 μM, less than 110 PM, less than 100 μM, or preferably less than 90 μM.

14. The method according to claim 9, wherein the liposomal composition comprises a glycan ligand formed by lipid attachment at the amine of the compound of formula 1

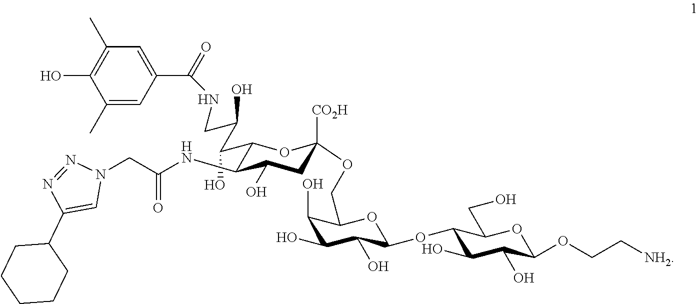

15. The method according to claim 14, wherein the glycan ligand is a compound of formula 3

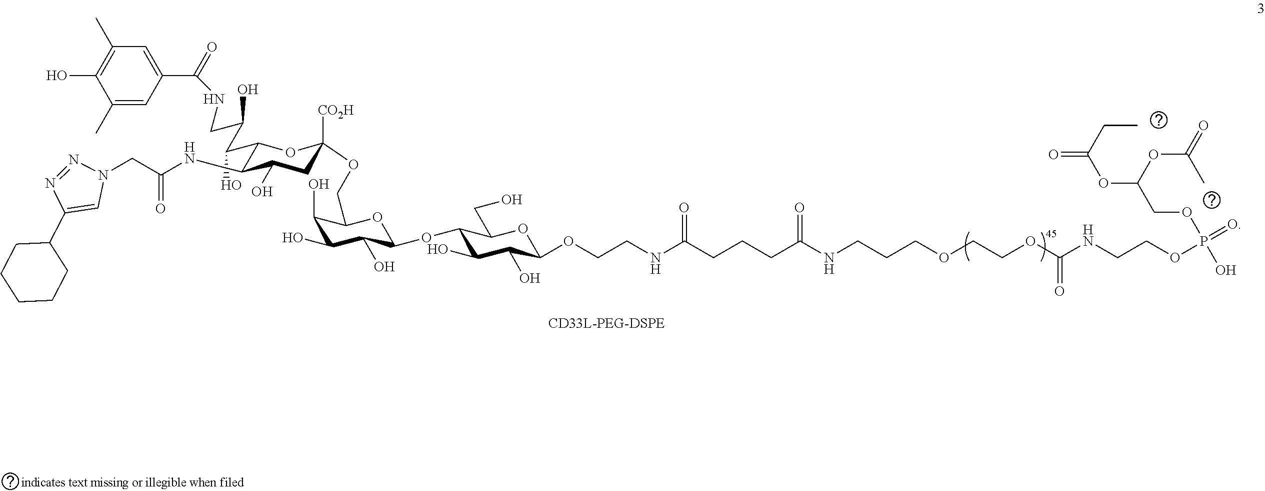

16. The method according to claim 9, wherein the liposomal composition is administered directly to the brain of the subject.

17. The method according to claim 16, wherein the liposomal composition is administered to the subject by intracerebroventricular injection or intraparenchymal injection.

18. (canceled)

19. (canceled)
